(19) United States
(12) Patent Application Publication
(10) Pub. No.: US 2020/0360452 A1
(43) Pub. Date: Nov. 19, 2020

(54) HEALTH BENEFICIAL COMPOSITION COMPRISING LACTOBACILLUS AND METHOD FOR THE PREPARATION THEREOF

(71) Applicant: VETCARE OY, Salo (FI)

(72) Inventors: Shea BEASLEY, Ojakkala (FI); Eila JÄRVENPÄÄ, Lieto (FI); Jarkko MÄKINEN, Helsinki (FI); Tuomo TUPASELA, Tarvasjoki (FI); Jaakko HIIDENHOVI, Forssa (FI); Kalevi HEINONEN, Hautjärvi (FI)

(73) Assignee: VETCARE OY, Salo (FI)

(21) Appl. No.: 16/959,892

(22) PCT Filed: Feb. 5, 2019
(86) PCT No.: PCT/FI2019/050080

§ 371 Date: Jul. 2, 2020

(30) Foreign Application Priority Data
Feb. 5, 2018 (FI) 20185099

(51) Int. Cl. A61K 35/747; A61K 33/06; A61K 36/45; A61K 36/73; A61K 36/82; A61K 47/36; A23K 10/18; A23K 50/40; A23K 20/24; A23K 10/30

(52) U.S. Cl. CPC A61K 35/747; A61K 33/06; A61K 36/45; A61K 36/73; A23K 10/30; A61K 47/36; A23K 10/18; A23K 50/40; A23K 20/24; A61K 36/82

(57) ABSTRACT

The present invention relates to a health beneficial composition for preventing and treating canine gastrointestinal and urinary tract disorders. The invention further relates also to a process for the manufacture of the health promoting composition, to dog food, and to the use of the health beneficial preparation for the manufacture of a pharmaceutical product or a dog food product for preventing and treating canine gastrointestinal and urinary tract disorders.

### FIELD OF THE INVENTION

The present invention relates to a health beneficial composition for preventing and/or treating gastrointestinal disorders and/or modifying urinary tract microbiota via gastro-intestine, particularly in dogs. The invention further relates to a method for the manufacture of said composition, to products comprising the composition and to use of the composition for the manufacture of pharmaceuticals and edible products for dogs.

### BACKGROUND OF THE INVENTION

Health beneficial lactic acid bacteria are widely used for enhancing the balance of beneficial and deleterious bacteria in the gastrointestinal tract of animals, such as dogs. Also, pet food comprising probiotic lactic acid bacteria has been suggested for improving health of the gastrointestinal tract and skin and/or coat system of cats and/or dogs, and ameliorating or reducing the effects of ageing.

Gastrointestinal and urinary tract disorders are very common among dogs. The traditional approach to the treatment of canine gastrointestinal problems relies on dietary modifications, antibiotic treatment, and specific anti-inflammatory and immunosuppressive drugs, either individually or combined. Many of the canine gastrointestinal as well as urinary tract disorders are treated with antibiotics, even when the diagnosis is uncertain or yet tentative. This treatment may involve even weeks of antibiotic therapy with several renewals. Due to increasing problems with antimicrobial resistance, alternative therapies involving treatment with probiotic bacteria, especially with lactic acid bacteria, have been suggested due to their health-conferring properties.

The indigestion of probiotic lactic acid bacteria has many benefits, such as modulation of the GI-tract, antagonism against pathogenic microbes, modulating GI and urinary tract microbiota, and maintaining the intestinal mucosal barrier.

Beasley S. et al, (Lactic acid bacteria isolated from canine faeces, Journal of Applied Microbiology, 101 (2006) 131-138) disclose the isolation and sequencing of lactic acid bacteria from the faeces of healthy dogs. Five of the strains, Lactobacillus fermentum, L. mucosae, L. rhamnosus, L. salivarius and Weissella confusa, were selected as candidate probiotics based on their frequency, quantity in faeces, growth density, acid tolerance and anti-microbial activity.

The research of Beasley et al was continued by Manninen T. et al. (Alteration of the Canine Small-Intestinal Lactic Acid Bacterium Microbiota by Feeding of Potential Probiotics, Applied and Environmental Microbiology, October 2006, p. 6539-6543) in an examination of the in vitro tolerances of the above-mentioned five candidate probiotic strains of lactic acid bacteria to canine jejunal chyme. The strains were fed twice a day mixed with dog food for 7 days to five permanently fistulated beagles. The strains were found to survive in and to dominate the jejunal chyme lactic acid microbiota during feeding and to have the ability to modify the intestinal microbiota.

In addition to use of probiotics, also other approaches to modify canine gut flora/microbiota have been disclosed. Oligosaccharides, such as inulin and various fructo-oligosaccharides have been reported to favour the growth of bifidobacteria and lactobacteria in the gastro-intestinal tract and decrease the growth of pathogens, such as Clostridium perfringens. EP 0 850 569 B1 discloses a cereal product useful as a pet food comprising a gelatinized starch matrix containing prebiotic oligosaccharide in the form of inulin, and optionally also prebiotic fructo-oligosaccharide. This product is said to have beneficial effect in the gastro-intestinal tract of the pet. When fed to dogs improved palatability, increased bifidobacteria counts, decreased C. perfringens counts, and decreased faecal pH, odour and volume were reported.

Also, probiotics combined with other potentially beneficial substances have been disclosed. WO 2007/076534 discloses a composition comprising at least one antioxidant such as vitamin E, vitamin C and/or β-carotene optionally in conjunction with one or more of a probiotic and a prebiotic. As suitable probiotics several species of Bifidobacterium and Lactobacillus are listed, oligosaccharides, galactans and β-glucans being mentioned as suitable prebiotics. The composition is stated to be useful for enhancing the balance of beneficial and deleterious bacteria in the gastrointestinal tract of an animal having a risk for inflammatory bowel disease, said animals including humans as well as avian, bovine, canine, equine, feline, hircine, murine, ovine and porcine animals.

US 2005/0175598 A1 discloses methods of use of probiotic Bifidobacteria, obtainable by isolation from resected and washed GI tract of mammals, preferably of dogs, in companion animals, these methods including treatment of immune system, weight control and body composition, urinary health, skin and coat diseases, and ageing. Said probiotics can be administered orally in viable or non-viable form, for example prepared into a composition for normal dietary intake such as kibbles and wet animal food, or to be used as a supplement, exemplified by biscuits, chews, treats, powders, suspensions, and capsules. As additional components the compositions may comprise protein, fat, carbohydrate, prebiotics, long-chain fatty acids, and zinc. Examples of prebiotics include oligosaccharides, fructo-oligosaccharides, galacto-oligosaccharides, xylo-oligosaccharides, and oligo derivatives of starch.

EP 1 290 136 B1 discloses six probiotic strains of lactic acid bacteria: feline Lactobacillus reuteri NCC2581, Lactobacillus reuteri NCC2592 and Lactobacillus rhamnosus NCC2583, and canine Lactobacillus reuteri NCC2603, Lactobacillus reuteri NCC2613 and Lactobacillus acidophilus NCC2628. Also disclosed is a method of preparing a dog or cat food composition including an additional step of incorporating the selected strain(s) into a dog or cat food composition. As suitable bacterial strains Lactobacillus reuteri, L. acidophilus, L. animalis, L. ruminis, L. johnsonii, L. casei, L. paracasei, L. rhamnosus, L. fermentum, Bifidobacterium sp., Enterococcus faecium, and Enterococcus sp. are listed. The pet food is intended for the health of the gastrointestinal tract and skin and/or coat system of cats and/or dogs, and ameliorating or reducing the effects of ageing. The pet food may contain, in addition to the bacteria strains and/or its fermented medium, a starch source, a protein source and lipid source, a prebiotic carbohydrate in an amount of less than about 20% by weight of the dried pet food, as well as long chain fatty acids, minerals and vitamins to supplement the pet food into a nutritionally complete product.

Probiotic products have typically limited stability and thereby the desired effects may be reduced or even lost.

Despite of the recent developments in the field, it is evident, that there still is a need for an improved dog-specific health beneficial composition, which can be used for preventing and treating a wide spectrum of canine gastrointestinal and urinal disorders, and secondary conditions originating from these disorders, where the use of antibiotics may even be avoided.

### OBJECT OF THE INVENTION

An object of the present invention is to provide a composition for preventing and/or treating gastrointestinal disorders and urinary tract infections particularly in dogs.

Another object of the present invention is to provide a composition for modulating canine gastrointestinal and urinary tract microbiota towards a healthy and stable microbiota.

Yet another object of the invention is a process for the manufacture of said composition.

Still another object of the present invention is the use of the composition for the manufacture of pharmaceutical and edible products for dogs.

Still another object of the present invention is a method for the manufacture of pharmaceutical and edible products for dogs comprising said composition.

The benefits of the present composition are seen especially in the improved stability and efficacy of the composition, which are particularly useful in the long-term treatment of chronic disorders, in disorders not responding to other therapies or to specific diet, and maintaining the health of the dog when improving the natural immunosuppressive status.

The characteristic features of the composition, the process for the manufacture of said composition, the pharmaceutical and edible products for dogs, and the use of the composition in the manufacture of a pharmaceutical compositions and edible products for dogs are disclosed in the claims.

### SUMMARY OF THE INVENTION

The invention is directed to a composition, which is suitable for preventing and treating canine gastrointestinal disorders and urinary tract infections, and secondary conditions originating therefrom. Particularly, the composition is useful for modulating canine gastrointestinal and urinary tract microbiota towards a healthy and stable microbiota.

Said composition comprises:
40-80 wt % of preparation A, comprising lyophilized dog-specific strains of lactic acid bacteria, where at least two of the strains belong to genus Lactobacillus, and at least one calcium source in an amount of 20-99 weight-% expressed as CaCO₃ of the dry weight of the preparation A,
20-60 wt % of preparation B, comprising 10-60 wt % of berry powder selected from bilberry powder, aronia powder, blackcurrant powder, bog bilberry powder, crowberry powder and combinations thereof, 10-60 wt % of berry powder selected from cranberry powder, red wine powder, lingonberry powder, raspberry powder and combinations thereof and 10-30 wt % of berry powder selected from sea buckthorn berry powder, saskatoon berry powder, cloudberry powder, arctic bramble powder, sorbus powder and combinations thereof.

The invention is also directed to a process for the manufacture of the composition. The process involves culturing either separately or together dog-specific strains of lactic acid bacteria, at least two of the strains belonging to genus Lactobacillus, to obtain lyophilized culture(s), and processing the obtained lyophilized culture(s), and a calcium source present in an amount of 20-99 weight-%, expressed as CaCO₃ of the dry weight of the preparation A, into a homogenous preparation A, mixing 40-80 wt % of preparation A with 20-60 wt % of preparation B comprising 10-60 wt % of berry powder selected from bilberry powder, aronia powder, blackcurrant powder, bog bilberry powder, crowberry powder and combinations thereof, 10-60 wt % of berry powder selected from cranberry powder, red wine powder, lingonberry powder, raspberry powder and combinations thereof and 10-30 wt % of berry powder selected from sea buckthorn berry powder, saskatoon berry powder, cloudberry powder, arctic bramble powder, sorbus powder and combinations thereof, whereby a composition is obtained.

The composition can be used for the prevention and treatment of canine gastrointestinal disorders and urinary tract infections, particularly for modulating canine gastrointestinal and urinary tract microbiota towards a healthy and stable microbiota, either as dry powder, mixed into an edible product for dogs, or formulated into a pharmaceutical formulation e.g. for oral administration, such as into granules, tablets, chewing snacks and fermented products for dogs.

In the following, the invention is illustrated by detailed description, and by examples without wishing to limit the invention thereto.

### DETAILED DESCRIPTION OF THE INVENTION

It has been surprisingly found that a composition with significantly improved stability and efficacy can be obtained by the present invention. Particularly, the stability of the lyophilized lactic acid bacteria strains can be improved significantly, and shelf-life of the products can be extended. It was particularly surprising that the berry powder mixture comprising 10-60 wt % of berry powder selected from bilberry powder, aronia powder, blackcurrant powder, bog bilberry powder, crowberry powder and combinations thereof, 10-60 wt % of berry powder selected from cranberry powder, red wine powder, lingonberry powder, raspberry powder and combinations thereof and 10-30 wt % of berry powder selected from sea buckthorn berry powder, saskatoon berry powder, cloudberry powder, arctic bramble powder, sorbus powder and combinations thereof, containing high amounts of antibacterial polyphenolic compounds, particularly a combination of bilberry, cranberry and sea buckthorn, do not reduce or prevent the activity of the specific lyophilized lactic acid bacteria strains. On the contrary, in addition to improved stability, also the efficacy of the probiotic composition is improved.

The specific combination of the berry species provides an optimum combination of polyphenols, which each are attached to the bacteria in different way, thereby preventing and inhibiting non-desired bacterial adherence to cell wall. Since microbes tend to migrate, it is essential to replace the inhibited bacteria with health-promoting strains, such as canine-derived lactic acid bacteria.

A specific mixture of polyphenols assures the effect on several undesired bacteria species, such as Escherichia coli, Enterococcus faecalis, Enterococcus sp, Proteus mirabilis, Staphylococcus intermedius, Staphylococcus sp (coagulase negative), Streptococcus bovis, Gram negative rod, Gram positive cocci, as well as unidentified microbiota. The polyphenol mixture combines high anthocyanine content of berries selected from bilberry, aronia, blackcurrant, bog bilberry, and crowberry and combinations thereof, preferably bilberries (typically at least 500 mg/100 g, based on dry weight, of anthocyanines in nordic billberries), with high proanthocyanidine content of berries selected from cranberry, red wine (Vitis vinifera grapes), lingonberry, raspberry and combinations thereof, preferably cranberry (typically at least 300 mg/100 g of proanthocyanidines, based on dry weight, in nordic cranberries), with proanthocyanidines of berries selected from sea buckthorn, saskatoon, cloudberry, arctic bramble, sorbus and combinations thereof, preferably sea buckthorn (typically in the range of 300-2500 mg/100 g of proanthocyanidines, based on dry weight, in nordic sea buckthorn species), and flavonoids and carotenoids contained therein. Sea buckthorn berry has an over pronounced taste and flavor, and it is easily rejected by dogs, therefore the amounts may be lower in the composition.

### Composition

The composition of the invention comprises:
40-80 wt % of preparation A, comprising lyophilized dog-specific strains of lactic acid bacteria, where at least two of the strains belong to genus Lactobacillus, and at least one calcium source in an amount of 20-99 weight-% expressed as CaCO₃ of the dry weight of the preparation A,
20-60 wt % of preparation B comprising 10-60 wt % of berry powder selected from bilberry powder, aronia powder, blackcurrant powder, bog bilberry powder, crowberry powder and combinations thereof, 10-60 wt % of berry powder selected from cranberry powder, red wine powder, lingonberry powder, raspberry powder and combinations thereof and 10-30 wt % of berry powder selected from sea buckthorn berry powder, saskatoon berry powder, cloudberry powder, arctic bramble powder, sorbus powder and combinations thereof.

In a preferable embodiment the composition comprises 45-75 wt % of preparation A and 25-55 wt % of preparation B.

In another preferable embodiment the composition comprises 50-70 wt % of preparation A and 30-50 wt % of preparation B.

In a preferable embodiment preparation B comprises 10-60 wt % of bilberry powder, 10-60 wt % of cranberry powder, and 10-30 wt % of sea buckthorn berry powder.

Optionally, the composition may additionally comprise 0.5-10 wt %, preferably 0.5-5 wt % of green tea powder.

The composition of the invention exerts beneficial effect to dogs suffering from a wide spectrum of gastrointestinal and/or urinary tract disorders of both known and unknown aetiology, and from secondary conditions deriving from these disorders.

The amount of each dog-specific strain of lactic acid bacteria may range between a minimum amount needed for a health beneficial effect, i.e. 1*10⁶ cfu/g, and 1*10¹³ cfu/g. In a preferable embodiment, in view of the effect and economical aspects, the amount of each dog-specific strain of lactic acid bacteria ranges from 1*10⁷ to 1*10¹⁰ cfu/g, preferably from 5*10⁸ to 2.5*10⁹ cfu/g of the preparation A. This concentration builds a sufficient amount of said strains to the GI tract to act beneficially. The dog-specific strains of lactic acid bacteria are incorporated into the preparation A as lyophilized cultures thus bringing residues of fermentation medium to the preparation. These residues act as protective agents to the lactic acid bacteria, and as initial growth material to the bacteria in the GI tract after digestion, either as such or pre-fermented by other microbes in the gut. These residues typically comprise up to 15 weight-% of the dry weight of the preparation A, preferably up to 5 weight-% of the dry weight of the preparation A. Preferably viable dog-specific strains of lactic acid bacteria are used. However, alternatively non-viable, inactivated strains of lactic acid bacteria may also be used.

Herein by the expression of “dog-specific strains of lactic acid bacteria” it is meant lactic acid producing bacteria isolated from canine faeces, canine intestines or intestinal fluids, or strains of lactic acid producing bacteria isolated from canine faeces, canine intestines or intestinal fluids. Advantageously at least one of the selected strains is resistant to antibiotics.

The dog-specific strains of lactic acid bacteria may be obtained by isolating different dog-specific strains of lactic acid bacteria from the faeces of healthy dogs, and selecting from the isolated strains at least two strains belonging to genus Lactobacillus. The isolation of the dog-specific strains of lactic acid bacteria from the faeces of healthy dogs may be performed as disclosed by Beasley et al. (Lactic acid bacteria isolated from canine faeces, Journal of Applied Microbiology, 101 (2006) 131-138). The selection criteria may include capability to grow in low pH (in pH 1-2), tolerance to bile acid and oxygen, resistance to some specific antibiotics, or antimicrobial activities towards some specific pathogens such as Micrococcus luteus, or certain species of Enterococcus and Clostridia recognised as opportunistic pathogens. Preferably, the chosen bacteria are not affected by protease treatment indicating either protease resistance or a non-protein nature of the antimicrobial substance.

In an embodiment, the composition and preparation A comprises two to five dog-specific strains of lactic acid bacteria belonging to genus Lactobacillus selected from Lactobacillus fermentum, Lactobacillus plantarum, Lactobacillus rhamnosus, Lactobacillus salivarius and Lactobacillus mucosae.

In an embodiment, the composition comprises two to five strains of lactic acid producing bacteria isolated from canine faeces, canine intestines or intestinal fluids, belonging to genus Lactobacillus, selected from Lactobacillus fermentum, Lactobacillus plantarum, Lactobacillus rhamnosus, Lactobacillus salivarius and Lactobacillus mucosae.

In an embodiment, the composition comprises
40-80 wt % of preparation A, comprising lyophilized strains of lactic acid producing bacteria isolated from canine faeces, canine intestines or intestinal fluids, where at least two of the strains belong to genus Lactobacillus and they are selected from Lactobacillus fermentum NCIMB 41636, Lactobacillus plantarum NCIMB 41638 and Lactobacillus rhamnosus NCIMB 41640, and at least one calcium source in an amount of 20-99 weight-% expressed as CaCO₃ of the dry weight of the preparation A,
20-60 wt % of preparation B, comprising 10-60 wt % of berry powder selected from bilberry powder, aronia powder, blackcurrant powder, bog bilberry powder, crowberry powder and combinations thereof, 10-60 wt % of berry powder selected from cranberry powder, red wine powder, lingonberry powder, raspberry powder and combinations thereof and 10-30 wt % of berry powder selected from sea buckthorn berry powder, saskatoon berry powder, cloudberry powder, arctic bramble powder, sorbus powder and combinations thereof.

In a preferable embodiment, the composition and preparation A contains Lactobacillus fermentum NCIMB 41636 and Lactobacillus plantarum NCIMB 41638 as the dog-specific strains of lactic acid bacteria belonging to genus Lactobacillus.

In another preferable embodiment, the composition and preparation A contains Lactobacillus fermentum NCIMB 41636, Lactobacillus plantarum NCIMB 41638 and Lactobacillus rhamnosus NCIMB 41640 as the dog-specific strains of lactic acid bacteria belonging to genus Lactobacillus.

These strains were deposited on 30 Jun. 2009 in the National Collections of Industrial, Food and Marine Bacteria (NCIMB).

Optionally the composition and preparation A may comprise additional dog-specific strains of lactic acid bacteria, other than those belonging to genus Lactobacillus, for example strains belonging to genus Pediococcus, such as P. acidolactici, or to genus Weissella, such as W. confusa and W. cibaria. Examples of preferable additional dog-specific strains of lactic acid bacteria other than Lactobacillus, are P. acidolactici NCIMB 41637 and W. confusa NCIMB 41639. These strains were deposited on 30 Jun. 2009 in the National Collections of Industrial, Food and Marine Bacteria (NCIMB).

The calcium source may be any calcium-containing substance acceptable for use in oral formulations for dogs. Non-limiting examples of calcium source useful in the invention are calcium carbonate, calcium ascorbate, calcium alginate, calcium stearoyl-2-actylate, calcium sorbate, calcium formate, calcium acetate, calcium propionate, calcium lactate, calcium citrate, calcium stearates, synthetic calcium silicate, calcium tetrahydrogendiorthophosphate, calcium hydrogen ortho-phosphate, calcium hydroxide, calcium oxide, dicalcium diphosphate, calcium gluconate, calcium sulphite, calcium hydrogensulphite, calcium aluminium silicate, calcium digluconate, calcium guanylate, calcium inosinate, calcium-5′-ribonucleotides, calcium malate, calcium tartrate, calcium dinatrium EDTA, mono and dicalcium diphosphate, (sodium)calcium polyphosphate, calcium chloride, calcium ferrocyanide, calcium orthophosphate, and combinations thereof.

Preferably the calcium source is calcium carbonate due to its well-accepted nature and its absorbability. In addition, calcium carbonate is cost effective compared to equivalent calcium sources.

Calcium absorption improves, particularly in the presence of prebiotics, increasing whole body mineral content. The amount of the calcium source ranges from 20 to 99 weight-%, preferably from 40 to 95 weight-%, and even more preferably from 60 to 90 weight-% expressed as CaCO₃ of the dry weight of the preparation. Calculated as Ca these weight ranges are 8-40 weight-%, 16-38 weight-% and 24-36 weight-%, respectively.

The preparation A may optionally comprise at least one additional prebiotic. By the expression “prebiotics” it is meant here non-digestible food/feed ingredients that beneficially affect the host by selectively stimulating the growth and/or activity of one or a limited number of bacteria in the intestines, and thus improve host health. Suitable prebiotics useful in the probiotic composition include soybean flour; psyllium, carob, gum arabic, guar gum, cassia, tamarind kernel, karaya gum, tragacanth gum, xanthan gum, gellan gum, tara gum; beta-glucan and hydrolysates thereof; oligosaccharides of oat; monosaccharides such as tagatose, and derivatives thereof; disaccharides such as lactose, lactulose, trehalose, melibiose, cellobiose, raffinose, stachyose, isomaltose, isomaltulose, and derivatives thereof; fructo-oligosaccharides, gluco-oligosaccharides, galacto-oligosaccharides, xylo-oligosaccharides, gentio-oligosaccharides, malto-oligosaccharides, isomalto-oligosaccharides, chito-oligosaccharides, manno-oligosaccharides, and derivatives thereof; poly- and oligosaccharides such as arabinogalactan, galactomannan, pectin, lignin, soybean hemicellulose, xylan, pullulan, inulin, arrow root, liquorice root, sugar beet pulp, tapioca, resistant starch of corn, barley, oat, and derivatives thereof; dextrins such as maltodextrins, cyclodextrins and derivatives thereof; processed Eucheuma seaweed, Irish moss; and any combinations thereof. In a preferable embodiment, carbohydrates, preferably sucrose or maltodextrin is used in preparation A.

Prebiotics promote the metabolism and growth of lactic acid bacteria in the GI tract, and alter the existing intestinal microbes towards favorable microbiota. The amount of the prebiotic(s) may range from 0.5 to 50 weight-%, preferably from 0.5 to 20 weight-% in preparation A.

Preparation B comprises 10-60 wt % of berry powder selected from bilberry powder, aronia powder, blackcurrant powder, bog bilberry powder, crowberry powder and combinations thereof, 10-60 wt % of berry powder selected from cranberry powder, red wine powder, lingonberry powder, raspberry powder and combinations thereof and 10-30 wt % of berry powder selected from sea buckthorn berry powder, saskatoon berry powder, cloudberry powder, arctic bramble powder, sorbus powder and combinations thereof.

In a preferable embodiment preparation B comprises 10-60 wt % of bilberry powder, 10-60 wt % of cranberry powder, and 10-30 wt % of sea buckthorn berry powder. Preferably, preparation B comprises 30-50 wt % of bilberry powder, 30-50 wt % of cranberry powder, and 15-25 wt % of sea buckthorn berry powder.

Optionally, the composition may additionally comprise 0.5-10 wt %, preferably 0.5-5 wt % of green tea powder.

The average particle size of bilberry powder is 90 wt-% less than 2 mm, preferably 90 wt-% less than 1.5 mm, particularly preferably 90 wt-% less than 1 mm.

The average particle size of cranberry powder is 90 wt-% less than 2 mm, preferably 90 wt-% less than 1.5 mm, particularly preferably 90 wt-% less than 1 mm.

The average particle size of sea buckthorn powder is 90 wt-% less than 2 mm, preferably 90 wt-% less than 1.5 mm, particularly preferably 90 wt-% less than 1 mm.

The bilberry may be selected from the species Vaccinium myrtillus L., Vaccinium uliginosum L., Vaccinium caespitosum Michx., Vaccinium deliciosum Piper, Vaccinium membranaceum and Vaccinium ovalifolium. Preferably nordic bilberry (Vaccinium myrtillus) is used due to its high proanthocyanidine content.

The cranberry may be selected from the species Vaccinium erythrocarpum, Vaccinium macrocarpum, Vaccinium microcarpum and Vaccinium oxycoccos.

Preferably nordic cranberry (Vaccinium oxycoccos) is used due to its high anthocyanine content.

The sea buckthorn may be selected from the species Hippophae goniocarpa, Hippophae gyantsensis, Hippophae litangensis Hippophae neurocarpa, Hippophae rhamnoides, Hippophae salicifolia and Hippophae tibetana.

Preferably common sea buckthorn (Hippophae rhamnoides) is used due to its high proanthocyanidine content, and due to flavonoids and carotenoids therein.

Preparation B comprises 2-15 wt % of polyphenolic compounds, preferably 3-10 wt %, particularly preferably 3-8 wt %, determined as gallic acid.

Preparation B comprises additionally fibers typically in the amount from 20 to 60 wt %.

The berry powders of preparation B act also as prebiotics, whereby it is optional to add an additional prebiotic in preparation A.

Preparation B is obtained by mixing bilberry powder, obtained from dried bilberries by grinding, cranberry powder obtained from dried cranberries by grinding, and sea buckthorn berry powder obtained from dried sea buckthorn berries by grinding.

Additionally, the composition may comprise at least one excipient, such as silicon dioxide, colloidal silicon dioxide, calcium silicate, magnesium silicate, magnesium trisilicate, talc, sodium aluminium silicate, potassium aluminium silicate, calcium aluminium silicate, bentonite, aluminium silicate, magnesium stearate, flavouring agents, and colouring agents. In a preferable embodiment silicon dioxide is used for providing improved flowability. Typically, the excipients are present in an amount ranging from 0 to 5 weight-%, preferably from 0.1 to 3 wt %, particularly preferably from 0.5 to 2 wt % of the dry weight of the composition.

Without wishing to be bound by any theory, it is believed that the benefits of the present composition are derived from the specific combination of the dog-specific strains of lactic acid bacteria, calcium and the specific berry powders. Lactobacillus sp. are known to be safe, and have shown to be able to colonise intestines, thus having longer wash-out period, and also to contribute to the colonisation of beneficial bacteria already present in the intestines of the subject being treated. The high Ca content produces a positive effect on the lumen stability possibly by affecting the interstices of intestinal epithelium and reducing leakage of fluids from the body into the intestines. The specific selected berry powders have a positive effect on inhibiting and preventing harmful bacterial adherence on canine urinary tract. The fibre contained in the berry powders has also proven to have an impact on gastrointestinal well-being such as preventing inflammatory bowel disease, gastric ulcer and colonic cancer together with health beneficial intestinal microbiota. The berry powders act as prebiotics in the composition and they also contribute to the ability of lactic acid bacteria to colonise the intestines after consumption. They alter selected intestinal microbiota, also the urinary tract by fermentation, reduce or even destroy the harmful bacteria, and therefore ensure the effectiveness of the probiotics. They may also act as enhancers of calcium absorption, and bind excessive liquid from stool.

### Process for the Manufacture of the Composition

The composition may be manufactured by combining preparation A with preparation B, with optional ingredients.

Said process comprises the steps, where
preparation A is manufactured by culturing either separately or together at least two dog-specific strains of lactic acid bacteria belonging to genus Lactobacillus, lyophilizing the cultures, and
the obtained culture(s), and a calcium source present in an amount of 20-99 weight-%, expressed as CaCO₃ of the dry weight of the preparation A, and optionally additional dog-specific strains of lactic acid bacteria, are processed into a homogenous preparation A,
mixing 40-80 wt % of the preparation A with 20-60 wt % of preparation B comprising 10-60 wt % of berry powder selected from bilberry powder, aronia powder, blackcurrant powder, bog bilberry powder, crowberry powder and combinations thereof, 10-60 wt % of berry powder selected from cranberry powder, red wine powder, lingonberry powder, raspberry powder and combinations thereof and 10-30 wt % of berry powder selected from sea buckthorn berry powder, saskatoon berry powder, cloudberry powder, arctic bramble powder, sorbus powder and combinations thereof, whereby a composition is obtained.

Preferably, preparation B comprising 10-60 wt % of bilberry powder, 10-60 wt % of cranberry powder, and 10-30 wt % of sea buckthorn berry powder, is used.

Optionally, additionally 0.5-10 wt %, preferably 0.5-5 wt % of green tea powder is mixed in the composition.

In an embodiment, the process comprises the steps, where
preparation A is manufactured by culturing, either separately or together, at least two strains of lactic acid producing bacteria isolated from canine faeces, canine intestines or intestinal fluids, selected from Lactobacillus fermentum NCIMB 41636, Lactobacillus plantarum NCIMB 41638 and Lactobacillus rhamnosus NCIMB 41640, lyophilizing the cultures, and
the obtained culture(s), and a calcium source present in an amount of 20-99 weight-%, expressed as CaCO₃ of the dry weight of the preparation A, and optionally additional strains of lactic acid producing bacteria isolated from canine faeces, canine intestines or intestinal fluids, are processed into a homogenous preparation A,
mixing 40-80 wt % of the preparation A with 20-60 wt % of preparation B comprising 10-60 wt % of berry powder selected from bilberry powder, aronia powder, blackcurrant powder, bog bilberry powder, crowberry powder and combinations thereof, 10-60 wt % of berry powder selected from cranberry powder, red wine powder, lingonberry powder, raspberry powder and combinations thereof and 10-30 wt % of berry powder selected from sea buckthorn berry powder, saskatoon berry powder, cloudberry powder, arctic bramble powder, sorbus powder and combinations thereof, whereby a composition is obtained.

The process involves manufacture of preparation A by culturing either separately or together at least two dog-specific strains of lactic acid bacteria belonging to genus Lactobacillus, lyophilizing the cultures and processing, suitably by mixing the obtained lyophilized culture(s), and a calcium source present in an amount of 20-99 weight-%, expressed as CaCO₃ of the dry weight of the preparation A, into a homogenous preparation A, mixing 40-80 wt % of preparation A with 20-60 wt % of preparation B comprising 10-60 wt % of bilberry powder, 10-60 wt % of cranberry powder, and 10-30 wt % of sea buckthorn berry powder, whereby a composition is obtained.

The strains of the lactic acid bacteria, calcium source and their amounts, the bilberry powder, cranberry powder, and sea buckthorn berry powder, and their amounts are selected as disclosed above.

Preparation B is obtained by mixing the berry powder obtained from dried bilberries, aronias, blackcurrants, bog bilberries, crowberries and combinations thereof by grinding, the berry powder obtained from dried cranberries, red wines, lingonberries, raspberries, and combinations thereof, and berry powder obtained from dried sea buckthorn berries, saskatoon berries, cloudberries, arctic brambles, sorbus berries and combinations thereof, by grinding. Dried berries can be obtained from fresh berries by drying using any suitable method, such as freeze-drying. Suitably the dried berries are grinded to average particle size of 90 wt-% less than 2 mm, preferably 90 wt-% less than 1.5 mm, particularly preferably 90 wt-% less than 1 mm. The grinding of the dried berries may be carried out using any suitable grinding apparatus, which can provide a fine powdery product.

In the manufacture of the preparation A, the dog-specific strains of lactic acid bacteria are cultured all together or preferably separately in liquid culture medium containing at least one carbon source and nitrogen source. Examples of suitable carbon sources include, without limitation, glucose, dextrose, and whey, alone or in combinations. Examples of suitable nitrogen sources include without limitation soybean flour, peptone, casein hydrolysate, meat extract, and yeast extract, dry yeast, non-specific protein-containing sources e.g. farmamedia, alone or in combinations. The dog-specific strains of lactic acid bacteria are cultured in the limited presence of oxygen without agitation or with gentle agitation until maximum cell density has been reached. Continuing any further will only lead to increased cell death. The pH of the cultures may range between 3.5 and 7, preferably it is between 4 and 6. The temperature may range between +25° C. and 37° C., preferably it is +30° C.±2° C. In this way, cell densities of at least 1×10¹ cfu/ml are obtained.

The cultivated cells are separated from the broth with any method including, without limitations, centrifuging, filtration or decantation. The cells separated from the fermentation broth are optionally washed by water, saline (0.9% NaCl) or with any suitable buffer. The wet cell mass obtained is dried by lyophilization. To enhance water adsorption there are several agents that are useful in drying while improving the stability of probiotics including, without any limitations, sucrose, maltodextrin, starch and other carbohydrates.

Several auxiliary substances may be used in the fermentation. They may be added to enhance the growth of lactobacilli by fermentation and the traces are advantageous to the final composition. The possible substances include e.g. a range of antifoam agents such as oil-based agents, silicone based materials, structol and, polypropylene or polyethylene glycols, without any limitation to those.

The processing of the obtained cultures and a calcium source into a homogenous preparation may involve any of the following in any order; lyophilising, centrifuging, filtering, drying, mixing, kneading, extruding, granulating, compressing, encapsulating, film-coating, and embedding or enclosing into control-released formulations.

In an embodiment, a first amount of at least one prebiotic is incorporated into the culture media of the dog-specific strains of lactic acid bacteria, the obtained cultures are optionally combined and washed, and lyophilised. They are subsequently mixed with the calcium source and optionally with a second amount of at least one prebiotic. By the expression “a first amount” it is meant any portion ranging from 0 to 100 weight-% of the total amount of the prebiotics, said first amount usually ranging from 0.001 to 30 weight-%, preferably being up to 15 weight-% of the dry weight of the preparation. By the expression “a second amount” it is meant the remaining portion of the total amount of the prebiotics not incorporated as said first amount. By incorporating a first amount of the prebiotics already into the culture media, partially fermented residues thereof may remain-depending on the selected processing steps—in the preparation A thus providing easily available material facilitating the colonisation of the strains.

Optionally, at least one excipient is mixed with the mixture of preparation A and preparation B. The excipient is selected from silicon dioxide, colloidal silicon dioxide, calcium silicate, magnesium silicate, magnesium trisilicate, talc, sodium aluminium silicate, potassium aluminium silicate, calcium aluminium silicate, bentonite, aluminium silicate, magnesium stearate, flavouring agents, and colouring agents. The at least one excipient is added to the mixture of preparation A and preparation B. In a preferable embodiment silicon dioxide is used for providing improved flowability. Typically, the excipients are present in an amount ranging from 0 to 5 weight-%, preferably from 0.1 to 3 wt %, particularly preferably from 0.5 to 2 wt % by dry weight of the composition.

The process of the invention may further comprise a step of formulating the composition into oral formulations in the form of powders, granules, pills, tablets, capsules, lozenges, dry products for reconstitution with water or other suitable carrier, aqueous or oily solutions or suspensions, gels, pastes, emulsions or syrups. The formulating may be carried out by conventional techniques, as described for example in “Remington: The Science and Practice of Pharmacy”, Lippincott, Williams and Wilkins Ed., December 2000, using suitable known binders, diluents, tabletting agents, lubricants, disintegrants, wetting agents, suspending agents, emulsifiers, non-aqueous carriers, preservatives, flavours or dyes as excipients and carriers.

In an embodiment the composition may be formulated with conventional ingredients used in edible dog products, into dog food, specialty dog food products, fresh food, sausages, frozen food, dry food pellets, kibbles, chunks, canned food, stews, pre-mixes, savoury sauce, biscuits, chewing snacks, treats, puppy milk replacers or fermented products, using methods well-known in the field, such as mixing, fermenting etc.

The composition of the invention may be used in such doses as to provide a daily intake within the following exemplary ranges:

Each probiotic strain: 2*10-2*10¹⁰ cfu/kg/day, preferably 1*10-5*10⁸ cfu/kg/day

Calcium: 40-198 mg/kg/day, preferably 80-190 mg/kg/day (expressed as calcium carbonate)

Prebiotics: 1-100 mg/kg/day, preferably 1-40 mg/kg/day.

The composition is suitably administered to dogs having body weight less than 15 kg (<15 kg dog range, typically small breeds) as daily doses from 0.5 to 8 g, a preferable dose being 4-6 g per<15 kg dog. Said doses provide 0.15-2.4 g, preferably 1.2-1.8 g of the preparation B (berry powder mixture), respectively.

The composition is suitably administered to dogs having body weight in the range of 15-30 kg (15-30 kg dog range, typically medium size breeds) as daily doses from 8 to 15 g, a preferable dose being 12-14 g per 15-30 kg dog. Said doses provide 2.4-4.5 g, preferably 3.6-4.2 g of the preparation B (berry powder mixture), respectively.

The composition is suitably administered to dogs having body weight more than 30 kg (>30 kg dog range, typically large breeds) as daily doses from 15 to 30 g, a preferable dose being 15-25 g per>30 kg dog. Said doses provide 4.5-9 g, preferably 4.5-7.5 g of the preparation B (berry powder mixture), respectively.

Optionally additional dog-specific strains of lactic acid bacteria and conventional excipients and carriers may be used in the composition.

Since the lactic acid bacteria in the composition is of canine origin, and only known berry powders and calcium sources acceptable in food/feed are used in the preparation, no adverse effects are anticipated. Typically, the composition is fed for periods of 5-10 days. Dogs may, however, benefit of a longer period, such as 30 days.

Due to its safety, the composition of the invention is particularly useful when treating chronic gastrointestinal and/or urinary tract disorders. In chronic disorders the probiotic preparation can be fed for substantially longer periods, such as for several months, continuously, or using intermitting administration.

Preferably the composition is formulated to provide easily dosed amount needed for dogs based on their weight, for example with a measuring spoon. Examples of the preferred forms include dry powders and granules.

The composition of the invention may also be incorporated into ready-to-use canine food such as fresh dog food, dog sausages, frozen dog food, canned dog food, stews, chunks, dry pellets, kibbles, treats or pre-mixes. In this case the amount of the preparation in the dog food is adjusted so that one meal or part of it, or all the meals, may be replaced by the dog food comprising the composition of the invention.

The composition of the invention may also be provided in a separate package, e.g. in a sachet, attached to the dog food package to be mixed with the dog food prior to ingestion. The composition of the invention may also be incorporated into canine specialty products such as fermented products, puppy milk replacers, capsules, savoury sauces, biscuits, chewing snacks or treats.

In case of a fermented product, the product may comprise water or milk, flavours, technical bacteria strains for fermenting, grains, and other conventional ingredients used in curdled milk, sour whole milk, yoghurt, and other fermented products.

In case of a puppy milk replacer the composition of the invention is incorporated into conventional puppy milk replacer ingredients either as a ready-to-use product, or the composition, provided separately e.g. in a sachet, is mixed with the puppy milk replacer just before use, or the composition is incorporated into a dry powder puppy milk replacer pre-mix to be recovered prior use with water, milk or other suitable liquid.

In case of capsules, the composition of the invention, e.g. in a form of a powder or suspension, is filled into conventional hard or soft capsules for example of gelatine.

In case of dog biscuits, chewing snacks, treats or savoury sauces, the composition of the invention is incorporated to biscuit, chewing snack, treat or savoury sauce ingredients, e.g. by mixing or by coating, as a ready-to-use product.

The composition of the invention is useful for preventing and treating a variety of canine gastrointestinal disorders and urinary tract infections, particularly for modulating canine gastrointestinal and urinary tract microbiota towards a healthy and stable microbiota. The combination of calcium and polyphenols inhibit leaking gut syndrome and pathogen adherence. Calcium enhances tight conjunction in the intestine inhibiting bacterial leakage from the intestine. Polyphenols prevent bacterial adherence. Health promoting lactic acid bacteria (LAB) modulate the intestinal microbiota allowing endogenous LAB to multiply.

In an embodiment, the composition is for use in preventing and treating canine gastrointestinal disorders.

In an embodiment, the composition is for use in preventing and treating canine urinary tract disorders.

The composition of the invention is particularly useful for treating small-intestine and urinary tract related disorders. Examples of such disorders are viral and bacterial infections, antibiotic-responsive enteropathy (ARE), irritable bowel syndrome (IBS), and inflammatory bowel disease (IBD). In addition, probiotic L. salivarius has been shown to clear pathogens in the GI-tract and thus, decreasing the risk of a pet dog acting as a symptomless pathogen carrier in the family. It is well known that family members, i.e. small children may receive pathogen infections from pets. By reducing pathogens in dogs with the composition, this risk may be reduced.

The composition of the invention is particularly useful for treating gastrointestinal disorders not responding to other treatment.

The composition of the invention is also useful for treating gastrointestinal disorders caused by unknown or multiple sources, or having alternating, or complex symptoms. It can be used alone, or simultaneously with a medication, also with some antibiotics. Using the probiotic preparation of the invention simultaneously with a medication known to cause gastrointestinal problems is particularly beneficial.

Other examples of preferable embodiments are the use before and during stressful situations, such as mating season, service, gestation, delivery, lactation, weaning and neonatal maternal separation. Gestating bitches may benefit from the composition especially through enhancement of the immune system, prevention of stress-related symptoms, and prevention of post-labour infections.

New-born and puppies may benefit from the composition especially through strengthening of natural microbial interaction in the GI-tract, enhancement of the immune system, suppressing of allergies, and avoiding puppy diarrhoea when changing diet to solid food.

Adult dogs may benefit from the composition especially through curing and prevention of gastrointestinal conditions such as antibiotic associated diarrhoea, prevention of allergies, prevention of infections such as ear, skin, vaginal, and urinary infections, maintenance of oral and dental hygiene, and prevention of stress-related symptoms.

Aging dogs may benefit from the composition especially through strengthening of natural microbial interaction in the GI-tract, enhancement of the immune system and maintenance of resistance to diseases, prevention of stress-related symptoms, and prevention of infections such as ear, skin, vaginal, and urinary infections.

The health beneficial composition of the invention may also be found beneficial in order to prevent gastrointestinal disorders when travelling by car, train or airplane, relocating, changing diet, visiting veterinary clinics and before/during hospitalisation due to surgical operations, and for hunting and competing dogs as well as during warm and damp seasons or dogs who swim. The health beneficial-composition of the invention maintains the healthy balance in the canine GI tract during severe training, competing, and rest periods.

Non-limiting examples of the primary and secondary disorders which may benefit from the use of the composition of the invention include inflammatory disorders, immunodeficiency, inflammatory bowel disease, irritable bowel syndrome, cancer (particularly those of the gastrointestinal and immune systems), diseases involving diarrhoea, antibiotic associated diarrhoea, appendicitis, autoimmune disorders, multiple sclerosis, Alzheimer's disease, amyloidosis, rheumatoid arthritis, arthritis, joint mobility, diabetes mellitus, insulin resistance, bacterial, viral and fungal infections, periodontal disease, diseases of oral cavity, urogenital disease, surgical associated trauma, surgical-induced metastatic disease, sepsis, weight loss, weight gain, excessive adipose tissue accumulation, anorexia, fever control, cachexia, wound healing, ulcers, gut barrier infection, allergy, asthma, respiratory disorders, circulatory disorders, coronary heart disease, anaemia, disorders of the blood coagulation system, renal disease, disorders of the central nervous system, hepatic disease, ischaemia, nutritional disorders, osteoporosis, endocrine disorders, epidermal disorders, and furunculosis. Preferred are treatment of the gastrointestinal tract and/or urinary tract, including treatment or prevention of diarrhoea; immune system regulation, preferably the treatment or prevention of autoimmune disease and inflammation; maintaining or improving the health of the skin and/or coat system, preferably treating or preventing atopic disease of the skin; maintaining or improving the health of the nails; ameliorating or reducing the effects of aging, including mental awareness and activity levels; and preventing weight loss during and following infection.

### EXAMPLES

### Example 1: Manufacture of the Composition

Selection of the Lactic Acid Bacteria Strains Lactobacillus fermentum, Lactobacillus plantarum and Lactobacillus rhamnosus were isolated from faeces of healthy canines by Beasley et al 2006, in the article referred to as LAB8 (L. fermentum), LAB9 (L. plantarum, previously identified as L. salivarius) and LAB11 (L. rhamnosus). Here, these strains are referred to by their deposit numbers NCIMB 41636, NCIMB 41638 and NCIM 41640, respectively. These bacteria have been demonstrated to survive low pH (pH 1) and can be cultured after collection from canine jejunum (Beasley et al 2006; Manninen et al, 2006). The strains alter the pre-existing intestinal microbiota facilitating the survival of the host specific lactic acid bacteria already present.

Intestinal modification in conjunction with antimicrobial activity enhance the probiotic nature of these strains. Due to this feature the strains can be given simultaneously with antibiotics to reduce antibiotic-induced diarrhoea, such as cephalosporins.

### Preparation A

### Culture Conditions

Lactobacillus fermentum NCIMB 41636, Lactobacillus plantarum NCIMB 41638 and Lactobacillus rhamnosus NCIMB 41640 were inoculated separately from the freshly prepared agar plate culture or from a freezed culture stock in 20% glycerol to the MRS (De Man, Rogosa & Sharpe)-medium (content: peptone (Bacto Peptone, Becton Dickinson) 10 g/l, meal extract (Organotechnie) 8 g/l, yeast extract (DSM Food Specialties) 4 g/l, dextrose 20 g/l, K₂HPO₄×3 H₂O 2.6 g/l, CH₃COONa×3H₂O 5 g/l, triammonium citrate 2 g/l, MgSO₄×7 H₂O 0.2 g/l and MnSO₄×1 H₂O 0.04 g/l and the cultivation was allowed to continue for 16-18 h at 30° C. without shaking.

The culture broth obtained was used to seed a 500 L of fermentation medium with 1% transferring rate. The production medium was as presented in Table 1:

TABLE 1

| Component | g/L |
|---|---|
| Glucose (Dextrose) | 24 |
| Soy Flour 7B | 30 |
| Yeast extract | 10 |
| K₂HPO₄ | 2.5 |
| Sodium acetate trihydrate | 7.5 |
| MnSO₄ × 1 H₂O | 0.1 |

As antifoam agent silicon based agent was used. The fermentation was carried out at 30° C. without aeration and with minimal agitation for one day. After reaching the OD600 value of 10, corresponding to the 6.0*10⁹; 4.0*10⁹; 4.0*10⁹ cfu/ml for Lactobacillus fermentum NCIMB 41636, Lactobacillus plantarum NCIMB 41638 and Lactobacillus rhamnosus NCIMB 41640, respectively, the cells were harvested by a separator (Seital SE 12 X). The wet cell masses of each Lactobacillus strain obtained from the 500 L fermentation culture broths were washed with water, supplemented with maltodextrin and dried by lyophilisation (Hetosicc Freeze dryer CD 15-1) for 3 days. Considering the value of cfu/mg, the yields after lyophilization for Lactobacillus fermentum NCIMB 41636, Lactobacillus plantarum NCIMB 41638 and Lactobacillus rhamnosus NCIMB 41640 were 90.1%, 47.1% and 45.6%, respectively. The residues of fermentation broth after washing contain minor quantities of soybean flour, e.g. 1-5 g/l and no other residues were found to be fully soluble component in the cultivation broth.

CaCO₃ and prebiotic (maltodextrin) were added to the lyophilized probiotics (Lactobacillus fermentum NCIMB 41636, Lactobacillus plantarum NCIMB 41638 and Lactobacillus rhamnosus NCIMB 41640) to obtain preparation A.

### Preparation B

Preparation B (Refinie) was obtained by mixing 40 wt-% of bilberry powder (Vaccinium myrtillus), 40 wt-% of cranberry powder (Vaccinium oxycoccos) and 20 wt-% of sea buckthorn powder (Hippophae rhamnoides). Preparation B contained 4.1-4.4 wt % of polyphenolic compounds. Preparation A, preparation B and SiO₂ were mixed to obtain the composition. In Table 2 examples of compositions 1-3 are presented.

TABLE 2

| | Lyophilized | | CaCO₃ g/wt-% in | Preparation | |
|---|---|---|---|---|---|
| | LABs | Maltodexrin | preparation A | B | SiO₂ |
| 1 | 290 g | — | 400 g/57% | 300 g | 10 g |
| 2 | 203 g | 87* g | 400 g/51% | 300 g | 10 g |
| 3 | 90 g | — | 400 g/80% | 500 g | 10 g |

*= in the probiotics mixture

### Example 2: Stability Tests on Composition 1 at +6° C. Temperature

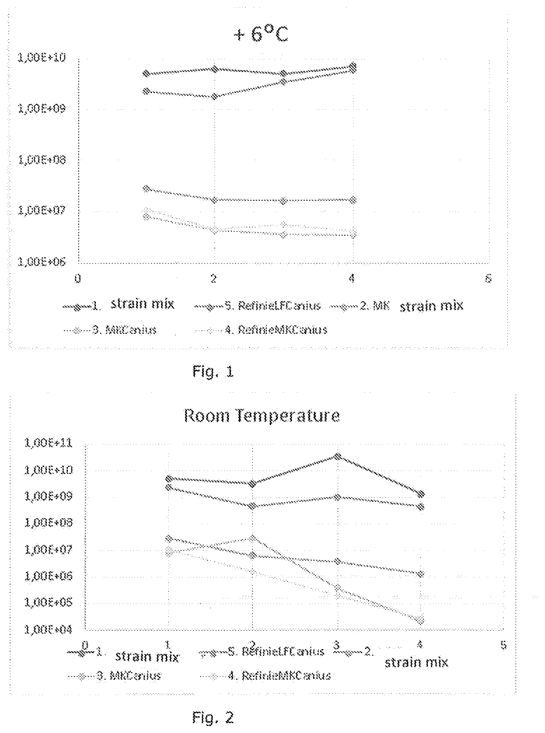

In Table 3, results of stability tests of lactic acid bacteria of lyophilized LAB strain mixture (sample 1), of preparation A as disclosed in Example 1 (sample 6), of composition 1 as disclosed in Example 1 containing lyophilized LABs (sample 5), of microcapsulated LAB strain mixture (sample 2), of preparation A as disclosed in Example 1, however containing microcapsulated LAB strain mixture (sample 3), and of composition 1 as disclosed in Example 1, however containing microcapsulated LAB strain mixture (sample 4). The microcapsulated LAB strain mixture contains the lactic acid bacteria in non-lyophilized microencapsulated form. The testing was carried out at +6° C. temperature for 6 months. The results of samples 1-5 are presented graphically in FIG. 1.

TABLE 3

| | | 2 months, | 4 months, | 6 months, |
|---|---|---|---|---|
| Sample | 0 month | +6° C. | +6° C. | +6° C. |
| 1. Lyophilized LAB | 5.10E+09 | 6.30E+09 | 5.10E+09 | 7.10E+09 |
| strain mix | | | | |
| 6. Preparation A | 1.3E+08 | 1.0E+08 | na | * |
| containing | | | | |
| lyophilized LABs | | | | |
| 5. Composition | 2.30E+09 | 1.80E+09 | 3.50E+09 | 5.80E+09 |
| containing | | | | |
| preparation A with | | | | |
| lyophilized LABs | | | | |
| and preparation B | | | | |
| 2. Microcapsu-lated | 2.80E+07 | 1.70E+07 | 1.60E+07 | 1.70E+07 |
| LAB strain mix | | | | |
| 3. Preparation A | 7.90E+06 | 4.30E+06 | 3.60E+06 | 3.40E+06 |
| containing | | | | |
| microcapsulated | | | | |
| LABs | | | | |
| 4. Composition | 1.10E+07 | 4.50E+06 | 5.60E+06 | 4.10E+06 |
| containing | | | | |
| preparation A with | | | | |
| microcapsulated | | | | |
| LABs and | | | | |
| preparation B | | | | |

*Results will be available in March 2018

From the results it can be seen that the preparation B increased significantly the stability of lyophilized lactic acid bacterial in the composition of the present invention, at +6° C., where it was significantly better than that with the composition containing microcapsulated LABs.

### Example 3: Stability Tests of Composition 1 at RT

In Table 4, results of stability tests of lactic acid bacteria of the lyophilized LAB strain mixture (sample 1), of preparation A as disclosed in Example 1 (sample 6), of composition 1 as disclosed in Example 1 containing lyophilized LABs (sample 5), of microcapsulated LAB strain mixture (sample 2), of preparation A as disclosed in Example 1, however containing microcapsulated LAB strain mixture (sample 3), and of composition 1 as disclosed in Example 1 however containing microcapsulated LAB strain mixture (sample 4). The microcapsulated LAB strain mixture contains the lactic acid bacteria in non-lyophilized microencapsulated form. The testing was carried out at RT (20±1° C.) for 6 months. The results of samples 1-5 are presented graphically in FIG. 2.

TABLE 4

| | | 2 months, | 4 months, | 6 months, |
|---|---|---|---|---|
| Sample | 0 months | RT | RT | RT |
| 1. Lyophilized LAB | 5.10E+09 | 3.20E+09 | 3.40E+10 | 1.30E+09 |
| strain mix | | | | |
| 5. Composition | 2.30E+09 | 4.50E+08 | 1.00E+09 | 4.30E+08 |
| containing | | | | |
| preparation A with | | | | |
| lyophilized LABs | | | | |
| and preparation B | | | | |
| 2. Microcapsulated | 2.80E+07 | 6.40E+06 | 3.60E+06 | 1.30E+06 |
| LAB strain mix | | | | |
| 3. Preparation A | 7.90E+06 | 2.80E+07 | 3.80E+05 | 2.00E+04 |
| containing | | | | |
| microcapsulated | | | | |
| LABs | | | | |
| 4. Composition | 1.10E+07 | 1.60E+06 | 1.90E+05 | 2.70E+04 |
| containing | | | | |
| preparation A with | | | | |
| microcapsulated | | | | |
| LABs and | | | | |
| preparation B | | | | |

From the results it can be seen that the preparation B increased significantly the stability of lyophilized lactic acid bacterial in the composition of the present invention, at RT, and it was significantly better than that with the composition containing microcapsulated LABs.

1. A composition comprising:
40-80 wt % of preparation A, comprising lyophilized dog-specific strains of lactic acid bacteria, where at least two of the strains belong to genus Lactobacillus, and at least one calcium source in an amount of 20-99 weight-% expressed as CaCO₃ of the dry weight of the preparation A,
20-60 wt % of preparation B, comprising 10-60 wt % of berry powder selected from bilberry powder, aronia powder, blackcurrant powder, bog bilberry powder, crowberry powder and combinations thereof, 10-60 wt % of berry powder selected from cranberry powder, red wine powder, lingonberry powder, raspberry powder and combinations thereof and 10-30 wt % of berry powder selected from sea buckthorn berry powder, saskatoon berry powder, cloudberry powder, arctic bramble powder, sorbus powder and combinations thereof.

2. The composition according to claim 1, wherein the composition comprises 45-75 wt % of preparation A and 25-55 wt % of preparation B.

3. The composition according to claim 1, wherein the composition comprises 50-70 wt % of preparation A and 30-50 wt % of preparation B.

4. The composition according claim 1, wherein the preparation B comprises 10-60 wt % of bilberry powder, 10-60 wt % of cranberry powder, and 10-30 wt % of sea buckthorn berry powder.

5. The composition according to claim 1, wherein the composition comprises 0.5-10 wt % of green tea powder.

6. The composition according to claim 1, wherein the amount of each dog-specific strain of lactic acid bacteria ranges between 1*10⁷-1*10¹⁰ cfu/g.

7. The composition according to claim 1, wherein the amount of each dog-specific strain of lactic acid bacteria ranges between 5*10⁸-2.5*10⁹ cfu/g.

8. The composition according to claim 1, wherein the amount of the calcium source ranges between 40-95 weight-% expressed as CaCO₃ of the dry weight of the preparation A.

9. The probiotic composition according to claim 1, wherein composition comprises prebiotic(s) in a range between 0.5-50 weight-% of the dry weight of the composition.

10. The composition according to claim 1, wherein the composition comprises two to five dog-specific strains of lactic acid bacteria belonging to genus Lactobacillus selected from Lactobacillus fermentum, Lactobacillus plantarum, Lactobacillus rhamnosus, Lactobacillus salivarius and Lactobacillus mucosae.

11. The composition according to claim 1, wherein said dog-specific strains of lactic acid bacteria belonging to genus Lactobacillus are Lactobacillus fermentum NCIMB 41636 and Lactobacillus plantarum NCIMB 41638.

12. The probiotic composition according to claim 1, wherein said dog-specific strains of lactic acid bacteria belonging to genus Lactobacillus are Lactobacillus fermentum NCMB 41636, Lactobacillus plantarum NCIMB 41638 and Lactobacillus rhamnosus NCIMB 41640.

13. The composition according to claim 1, wherein the calcium source is selected from calcium carbonate, calcium ascorbate, calcium alginate, calcium stearoyl-2-lactylate, calcium sorbate, calcium formate, calcium acetate, calcium propionate, calcium lactate, calcium citrate, calcium stearates, synthetic calcium silicate, calcium tetrahydrogen-diorthophosphate, calcium hydrogen-orthophosphate, calcium hydroxide, calcium oxide, dicalcium diphosphate, calcium gluconate, calcium sulphite, calcium hydrogensulphite, calcium aluminium silicate, calcium digluconate, calcium guanylate, calcium inosinate, calcium-5′-ribonucleotides, calcium malate, calcium tartrate, calcium dinatrium EDTA, mono and dicalciumdiphosphate, (sodium)calciumpolyphosphate, calcium chloride, calcium ferrocyanide, calcium orthophosphate, or combinations thereof.

14. The composition according to claim 1, wherein bilberry is selected from the species Vaccinium myrtillus L., Vaccinium uliginosum L., Vaccinium caespitosum Michx., Vaccinium deliciosum Piper, Vaccinium membranaceum and Vaccinium ovalifolium.

15. The composition according to claim 1, wherein cranberry is selected from the species Vaccinium erythrocarpum, Vaccinium macrocarpum, Vaccinium microcarpum and Vaccinium oxycoccos.

16. The composition according to claim 1, in that wherein sea buckthorn is selected from the species Hippophae goniocarpa, Hippophae gyantsensis, Hippophae litangensis Hippophae neurocarpa, Hippophae rhamnoides, Hippophae salicifolia and Hippophae tibetana.

17. The composition according to claim 1, wherein said at least one prebiotic is selected from soybean flour; psyllium, carob, gum arabic, guar gum, cassia, tamarind kernel, karaya gum, tragacanth gum, xanthan gum, gellan gum, tara gum; beta-glucan and hydrolysates thereof, oligosaccharides of oat; monosaccharides such as tagatose, and derivatives thereof; disaccharides such as lactose, lactulose, trehalose, melibiose, cellobiose, raffinose, stachyose, isomaltose, isomaltulose, and derivatives thereof; fructo-oligosaccharides, gluco-oligosaccharides, galacto-oligosaccharides, xylo-oligosaccharides, gentio-oligosaccharides, malto-oligosaccharides, isomalto-oligosaccharides, chito-oligosaccharides, manno-oligosaccharides, and derivatives thereof; poly- and oligosaccharides such as arabinogalactan, galactomannan, pectin, lignin, soybean hemicellulose, xylan, pullulan, inulin, arrow root, liquorice root, sugar beet pulp, tapioca, resistant starch of corn, barley, oat, and derivatives thereof; dextrins such as maltodextrins, cyclodextrins and derivatives thereof; processed Eucheuma seaweed, Irish moss; and any combinations thereof.

18. The c composition according to claim 1, wherein the composition comprises at least one excipient, preferably silicon dioxide, colloidal silicon dioxide, calcium silicate, magnesium silicate, magnesium trisilicate, talc, sodium aluminum silicate, potassium aluminum silicate, calcium aluminum silicate, bentonite, aluminum silicate, magnesium stearate, flavoring agent or coloring agent.

19. The composition according to claim 1, wherein the composition is in the form of dry powders, granules, pills, tablets, capsules, lozenges, dry products for reconstitution with water or other suitable carrier, aqueous or oily solutions or suspensions, gels, pastes, emulsions or syrups.

20. An edible product for dogs, wherein the edible products for dogs contains the composition according to claim 1, and it is formulated into dog food, specialty dog food products, fresh food, sausages, frozen food, dry food pellets, kibbles, chunks, canned food, stews, pre-mixes, savoury sauce, biscuits, chewing snacks, treats, puppy milk replacers or fermented products.

21. A process for the manufacture of the composition according to claim 1, wherein the process comprises the steps, where
preparation A is manufactured by culturing, either separately or together, at least two dog-specific strains of lactic acid bacteria belonging to genus Lactobacillus, lyophilizing the cultures, and
the obtained culture(s), and a calcium source present in an amount of 20-99 weight-%, expressed as CaCO₃ of the dry weight of the preparation A, and optionally additional dog-specific strains of lactic acid bacteria, are processed into a homogenous preparation A,
mixing 40-80 wt % of the preparation A with 20-60 wt % of preparation B comprising 10-60 wt % of berry powder selected from bilberry powder, aronia powder, blackcurrant powder, bog bilberry powder, crowberry powder and combinations thereof, 10-60 wt % of berry powder selected from cranberry powder, red wine powder, lingonberry powder, raspberry powder and combinations thereof and 10-30 wt % of berry powder selected from sea buckthorn berry powder, saskatoon berry powder, cloudberry powder, arctic bramble powder, sorbus powder and combinations thereof, whereby a composition is obtained.

22. The process according to claim 21, wherein the preparation B comprises 10-60 wt % of bilberry powder, 10-60 wt % of cranberry powder, and 10-30 wt % of sea buckthorn berry powder.

23. The process according to claim 21, wherein 0.5-10 wt % of green tea powder is mixed in the composition.

24. The process according to claim 21, wherein the preparation A and the preparation B are mixed with at least one excipient, preferably silicon dioxide, colloidal silicon dioxide, calcium silicate, magnesium silicate, magnesium trisilicate, talc, sodium aluminum silicate, potassium aluminum silicate, calcium aluminum silicate, bentonite, aluminum silicate, magnesium stearate, flavoring agent or coloring agent.

25. The process according to claim 21, wherein bilberry is selected from the species Vaccinium myrtillus L., Vaccinium uliginosum L., Vaccinium caespitosum Michx., Vaccinium deliciosum Piper, Vaccinium membranaceum and Vaccinium ovalifolium.

26. The process according to claim 21, wherein cranberry is selected from the species Vaccinium erythrocarpum, Vaccinium macrocarpum, Vaccinium microcarpum and Vaccinium oxycoccos.

27. The process according to claim 21, wherein sea buckthorn is selected from the species Hippophae goniocarpa, Hippophae gyantsensis, Hippophae litangensis Hippophae neurocarpa, Hippophae rhamnoides, Hippophae salicifolia and Hippophae tibetana.

28. The process of claim 21, wherein it further comprises a step of formulating the composition into oral formulations in the form of powders, granules, pills, tablets, capsules, lozenges, dry products for reconstitution with water or other suitable carrier, aqueous or oily solutions or suspensions, gels, pastes, emulsions or syrups, or into edible dog product selected from dog food, specialty dog food products, fresh food, sausages, frozen food, dry food pellets, kibbles, chunks, canned food, stews, pre-mixes, savoury sauce, biscuits, chewing snacks, treats, puppy milk replacers or fermented products.

29. Use of a health promoting composition of claim 1, for the manufacture of a pharmaceutical product or a dog food product for preventing and treating canine gastrointestinal and urinary tract disorders.
